(19) United States
(12) Patent Application Publication
(10) Pub. No.: US 2005/0249812 A1
(43) Pub. Date: Nov. 10, 2005

(54) Micronized organic preservative formulations

(76) Inventors: Robert M. Leach, Grand Island, NY (US); Jun Zhang, Getzville, NY (US)

Correspondence Address: HODGSON RUSS LLP, ONE M & T PLAZA, SUITE 2000, BUFFALO, NY, 14203-2391, US

(21) Appl. No.: 11/116,152

(22) Filed: Apr. 27, 2005

Related U.S. Application Data
(60) Provisional application No. 60/565,585, filed on Apr. 27, 2004.

(51) Int. Cl. B32B 21/02; A61K 31/4178

(52) U.S. Cl. 424/489; 514/397; 428/292.4

(57) ABSTRACT

A wood preservative composition comprising micronized particles of organic biocide is provided. The composition comprises a dispersion comprising particles in the range of 0.001 to 25 microns. Also provided is a method for the application of the preservative composition to wood. In addition, wood products which have been treated with the preservative composition are also provided.

### CROSS REFERENCE TO RELATED APPLICATION

Applicant hereby claims priority to U.S. Provisional Application No. 60/565,585, filed on Apr. 27, 2004, which is incorporated herein by reference.

### BACKGROUND

Wood preserving compositions are used for preserving wood and other wood-based materials, such as paper, particleboard, wood composites, plastic lumbers, rope, etc., against organisms which destroy wood. Many conventional wood preserving compositions contain water insoluble organic biocides. Heretofore, organic biocides such as insecticides, fungicides, moldicides, algaecides, bactericides, etc. have been dissolved in organic carriers prior to use, often with the additional step of emulsification in water by the use of various surfactants.

Many of the organic biocides currently in use have very low water solubility and therefore, solubilizing agents or surfactants such as emulsifying agents, wetting agents, etc. are added in order to give a product that is suitable for the treatment of wood or other cellulose substrates. However, solubilizing agents or surfactants, etc. are costly and the use of these products may also result in enhanced leaching of organic biocide upon exposure of treated wood to moisture. It is thought that the enhanced leaching is due to the fact that solubilizing agents, surfactants, emulsifying agents, wetting agents, etc. remain in the wood after treatment. Upon exposure to moisture, the biocides are solubilized, and they wash out of the wood.

Excessive leaching of organic biocides from the treated wood or other cellulose substrates can result in field performance problems or environmental issues. However, despite the efforts of many inventors, there remains a need for organic preservative systems which are do not require organic solvents, which are suitable for use to treat wood and cellulose-based materials, yet having only low levels of leaching, if any, upon exposure of treated materials to the environment. This need is satisfied by the compositions disclosed herein.

### SUMMARY OF THE INVENTION

Disclosed herein is a micronized organic wood preservative composition and method for its use to treat cellulosic materials, particularly wood.

Current technology typically requires the addition of organic solvents, emulsifying agents, etc. Disadvantages of the typical approach used in the art include increased cost, odor, residue bleeding, environmental damage and harmful exposure to leached biocide.

With the inventive compositions disclosed herein, organic solvents are not required, thus reducing cost and odors. Furthermore, leaching of the organic biocide from treated materials is reduced relative to non-micronized or solubilized compositions currently used in the art, thus reducing environmental and exposure risks.

The composition comprises micronized organic biocides with little or no water solubility. The composition may additionally comprise water soluble organic biocides, as well as inorganic biocides which are either solvated or present as micronized particles. The term “micronized” as used herein means particles which have long axis dimensions in the range of from 0.001 to 25 microns.

Also provided is a method for the treatment of wood or wood product with the compositions of the present invention. In one embodiment, the method comprises the steps of 1) providing a mixture comprising micronized organic biocide particles in an aqueous carrier, such as in the form of a dispersion, emulsion, suspension, or other particle/carrier combination, and 2) applying the particles to a wood or wood product. In a further embodiment, the organic biocides are prepared by the grinding of the organic biocide, optionally in non-micronized particulate form, in wetting agents and/or dispersants such that the biocide is reduced to the form of micronized particles. When such a composition is used for preservation of wood, there is minimal leaching of the organic biocide from wood as described herein.

### BRIEF DESCRIPTION OF THE FIGURES

FIG. 1 depicts the anatomy of coniferous wood. A: Resin canal; B: Earlywood tracheids; C: Latewood tracheids; D: Bordered pits.

FIG. 2 depicts the border pit structure for coniferous woods. RIGHT: Microscopic view of the cross section of a bordered pit; LEFT: Torus in top view. The torus is supported by a net of radial fibril membrane, also called the margo. The flow of fluids between two tracheids through such a membrane is restricted by the size of the membrane openings. A: Pit aperture; B: Torus; C: Margo (microfibrils); D: Pit border

### DETAILED DESCRIPTION OF THE INVENTION

Unless stated otherwise, such as in the examples, all amounts and numbers used in this specification are intended to be interpreted as modified by the term “about”. Likewise, elements or compounds identified in this specification, unless stated otherwise, are intended to be non-limiting and representative of other elements or compounds generally considered by those skilled in the art as being within the same family of elements or compounds. Also, the term “organic biocide,” unless specifically stated otherwise, is intended to refer to fungicides, insecticides, moldicides, algaecides, bactericides or any other organic compound which serves as a biocidal agent.

In one embodiment, the organic biocides are azoles, carbamates, isothiazolinones, thiocyanates, sulfenamides, quaternary phosphonium compounds, quaternary ammonium compounds, nitrites, pyridines, etc. or mixtures thereof. The compositions contain micronized particles. Additionally, the organic biocides exhibit a low solubility in water. A solubility which is at most 0.5 g of biocide per 100 grams of water is preferred.

The micronized organic biocide can be obtained by grinding the organic biocides, optionally wetted or present as a dispersion, to the desired particle size using a grinding mill. Other particulating methods known in the art can also be used, such as high speed, high shear mixing or agitation. The resulting particulate organic biocide can be mixed with water or other aqueous liquid carrier to form a solution of dispersed biocide particles. Optionally, the solution can comprise a thickener, such as, for example, a cellulose derivative, as is known in the art. The solution can, optionally, additionally comprise other biocides, organic or inorganic, micronized if desired, to produce a formulation suitable for the preservation of wood and other cellulose-based materials.

Examples of the water insoluble organic fungicides, insecticides, moldicides, bactericides, algaecides, etc., which can be used in the compositions and methods of the present invention include azoles, carbamates, isothiazolinones, thiocyanates, sulfenamides, quaternary phosphonium compounds, quaternary ammonium compounds, nitriles, pyridines, and mixtures or synergistic mixtures thereof. Some non-limiting examples of suitably water insoluble organic biocides follow. Those skilled in the art will recognize that organic biocides other than those explicitly mentioned herein may be suitably insoluble for use in the compositions and methods of the present invention.

Examples of organic biocides useful for the present invention are provided in Tables 1, 2 and 3.

TABLE 1
Aliphatic Nitrogen Fungicides
butylamine; cymoxanil; dodicin; dodine; guazatine; iminoctadine
Amide Fungicides
carpropamid; chloraniformethan; cyazofamid; cyflufenamid; diclocymet; ethaboxam;
fenoxanil; flumetover; furametpyr; prochloraz; quinazamid; silthiofam; triforine;
benalaxyl; benalaxyl-M; furalaxyl; metalaxyl; metalaxyl-M; pefurazoate;
benzohydroxamic acid; tioxymid; trichlamide; zarilamid; zoxamide; cyclafuramid;
furmecyclox dichlofluanid; tolylfluanid; benthiavalicarb; iprovalicarb; benalaxyl;
benalaxyl-M; boscalid; carboxin; fenhexamid; metalaxyl; metalaxyl-M; metsulfovax;
ofurace; oxadixyl; oxycarboxin; pyracarbolid; thifluzamide; tiadinil; benodanil;
flutolanil; mebenil; mepronil; salicylanilide; tecloftalam fenfuram; furalaxyl; furcarbanil; methfuroxam;
flusulfamide
Antibiotic Fungicides
aureofungin; blasticidin-S; cycloheximide; griseofulvin; kasugamycin; natamycin;
polyoxins; polyoxorim; streptomycin; validamycin; azoxystrobin; dimoxystrobin;
fluoxastrobin; kresoxim-methyl; metominostrobin; orysastrobin; picoxystrobin;
pyraclostrobin; trifloxystrobin
Aromatic Fungicides
biphenyl; chlorodinitronaphthalene; chloroneb; chlorothalonil; cresol; dicloran;
hexachlorobenzene; pentachlorophenol; quintozene; sodium pentachlorophenoxide; tecnazene
Benzimidazole Fungicides
benomyl; carbendazim; chlorfenazole; cypendazole; debacarb; fuberidazole;
mecarbinzid; rabenzazole; thiabendazole
Benzimidazole Precursor Fungicides
furophanate; thiophanate; thiophanate-methyl
Benzothiazole Fungicides
bentaluron; chlobenthiazone; TCMTB
Bridged Diphenyl Fungicides
bithionol; dichlorophen; diphenylamine
Carbamate Fungicides
benthiavalicarb; furophanate; iprovalicarb; propamocarb; thiophanate; thiophanate-
methyl; benomyl; carbendazim; cypendazole; debacarb; mecarbinzid; diethofencarb,
iodopropynyl butylcarbamate
Conazole Fungicides
climbazole; clotrimazole; imazalil; oxpoconazole; prochloraz; triflumizole;
azaconazole; bromuconazole; cyproconazole; diclobutrazol; difenoconazole;
diniconazole; diniconazole-M; epoxiconazole; etaconazole; fenbuconazole;
fluquinconazole; flusilazole; flutriafol; furconazole; furconazole-cis hexaconazole;
imibenconazole; ipconazole; metconazole; myclobutanil; penconazole; propiconazole;
prothioconazole; quinconazole; simeconazole; tebuconazole; tetraconazole;
triadimefon; triadimenol; triticonazole; uniconazole; uniconazole-P
Dicarboximide Fungicides
famoxadone; fluoroimide; chlozolinate; dichlozoline; iprodione; isovaledione;
myclozolin; procymidone; vinclozolin; captafol; captan; ditalimfos; folpet; thiochlorfenphim
Dinitrophenol Fungicides
binapacryl; dinobuton; dinocap; dinocap-4; dinocap-6; dinocton; dinopenton;
dinosulfon; dinoterbon; DNOC
Dithiocarbamate Fungicides
azithiram; carbamorph; cufraneb; cuprobam; disulfiram; ferbam; metam; nabam;
tecoram; thiram; ziram; dazomet; etem; milneb; mancopper; mancozeb; maneb;
metiram; polycarbamate; propineb; zineb
Imidazole Fungicides
cyazofamid; fenamidone; fenapanil; glyodin; iprodione; isovaledione; pefurazoate;
triazoxide
Morpholine Fungicides
aldimorph; benzamorf; carbamorph; dimethomorph; dodemorph; fenpropimorph;
flumorph; tridemorph
Organophosphorus Fungicides
ampropylfos; ditalimfos; edifenphos; fosetyl; hexylthiofos; iprobenfos; phosdiphen;
pyrazophos; tolclofos-methyl; triamiphos
Oxathiin Fungicides
carboxin; oxycarboxin
Oxazole Fungicides
chlozolinate; dichlozoline; drazoxolon; famoxadone; hymexazol; metazoxolon;
myclozolin; oxadixyl; vinclozolin
Pyridine Fungicides
boscalid; buthiobate; dipyrithione; fluazinam; pyridinitril; pyrifenox; pyroxychlor;
pyroxyfur
Pyrimidine Fungicides
bupirimate; cyprodinil; diflumetorim; dimethirimol; ethirimol; fenarimol; ferimzone;
mepanipyrim; nuarimol; pyrimethanil; triarimol
Pyrrole Fungicides
fenpiclonil; fludioxonil; fluoroimide
Quinoline Fungicides
ethoxyquin; halacrinate; 8-hydroxyquinoline sulfate; quinacetol; quinoxyfen
Quinone Fungicides
benquinox; chloranil; dichlone; dithianon
Quinoxaline Fungicides
chinomethionat; chlorquinox; thioquinox
Thiazole Fungicides
ethaboxam; etridiazole; metsulfovax; octhilinone; thiabendazole; thiadifluor; thifluzamide
Thiocarbamate Fungicides
methasulfocarb; prothiocarb
Thiophene Fungicides
ethaboxam; silthiofam
Triazine Fungicides
anilazine
Triazole Fungicides
bitertanol; fluotrimazole; triazbutil
Urea Fungicides
bentaluron; pencycuron; quinazamid
Other Fungicides
acibenzolar acypetacs allyl alcohol benzalkonium chloride benzamacril bethoxazin
carvone chloropicrin DBCP dehydroacetic acid diclomezine diethyl pyrocarbonate
fenaminosulf fenitropan fenpropidin formaldehyde furfural hexachlorobutadiene
iodomethane isoprothiolane methyl bromide methyl isothiocyanate metrafenone
nitrostyrene nitrothal-isopropyl OCH 2 phenylphenol phthalide piperalin probenazole
proquinazid pyroquilon sodium orthophenylphenoxide spiroxamine sultropen thicyofen tricyclazole;
chitin; chitosan; 4-cumylphenol, , 4-alpha-cumylphenol.

Examples of useful organic insecticides are shown in Table 2:

TABLE 2
Antibiotic Insecticides
allosamidin; thuringiensin; spinosad; abarmectin; doramectin; emamectin eprinomectin;
ivermectin; selamectin; milbemectin; milbemycin oxime; moxidectin
Botanical Insecticides
anabasine; azadirachtin; d-limonene; nicotine; pyrethrins cinerins; cinerin I; cinerin II; jasmolin I;
jasmolin II; pyrethrin I; pyrethrin II; quassia; rotenone; ryania sabadilla
Carbamate Insecticides
bendiocarb; carbaryl; benfuracarb; carbofuran; carbosulfan; decarbofuran;
furathiocarb; dimetan; dimetilan; hyquincarb; pirimicarb; alanycarb; aldicarb;
aldoxycarb; butocarboxim; butoxycarboxim; methomyl; nitrilacarb; oxamyl;
tazimcarb; thiocarboxime; thiodicarb; thiofanox; allyxycarb aminocarb; bufencarb;
butacarb; carbanolate; cloethocarb; dicresyl; dioxacarb; EMPC; ethiofencarb;
fenethacarb; fenobucarb; isoprocarb; methiocarb; metolcarb; mexacarbate; promacyl;
promecarb; propoxur; trimethacarb; XMC; xylylcarb
Dinitrophenol Insecticides
dinex; dinoprop; dinosam; DNOC; cryolite; sodium hexafluorosilicate; sulfluramid
Formamidine Insecticides
amitraz; chlordimeform; formetanate; formparanate
Fumigant Insecticides
acrylonitrile; carbon disulfide; carbon tetrachloride; chloroform; chloropicrin; para-
dichlorobenzene; 1,2-dichloropropane; ethyl formate; ethylene dibromide; ethylene
dichloride; ethylene oxide; hydrogen cyanide; iodomethane; methyl bromide;
methylchloroform; methylene chloride; naphthalene; phosphine; sulfuryl fluoride;
tetrachloroethane
Insect Growth Regulators
bistrifluron; buprofezin; chlorfluazuron; cyromazine; diflubenzuron; flucycloxuron;
flufenoxuron; hexaflumuron; lufenuron; novaluron; noviflumuron; penfluron;
teflubenzuron; triflumuron; epofenonane; fenoxycarb; hydroprene; kinoprene;
methoprene; pyriproxyfen; triprene; juvenile hormone I; juvenile hormone II; juvenile
hormone III; chromafenozide; halofenozide; methoxyfenozide; tebufenozide; α-
ecdysone; ecdysterone; diofenolan; precocene I; precocene II; precocene III;
dicyclanil
Nereistoxin Analogue Insecticides
bensultap; cartap; thiocyclam; thiosultap; flonicamid; clothianidin; dinotefuran;
imidacloprid; thiamethoxam; nitenpyram nithiazine; acetamiprid; imidacloprid;
nitenpyram; thiacloprid
Organochlorine Insecticides
bromo-DDT; camphechlor; DDT; pp′-DDT; ethyl-DDD; HCH; gamma-HCH;
lindane; methoxychlor; pentachlorophenol; TDE; aldrin; bromocyclen; chlorbicyclen;
chlordane; chlordecone; dieldrin; dilor; endosulfan; endrin; HEOD; heptachlor;
HHDN; isobenzan; isodrin; kelevan; mirex
Organophosphorus Insecticides
bromfenvinfos; chlorfenvinphos; crotoxyphos; dichlorvos; dicrotophos;
dimethylvinphos; fospirate; heptenophos; methocrotophos; mevinphos;
monocrotophos; naled; naftalofos; phosphamidon; propaphos; schradan; TEPP;
tetrachlorvinphos; dioxabenzofos; fosmethilan; phenthoate; acethion; amiton;
cadusafos; chlorethoxyfos; chlormephos; demephion; demephion-O; demephion-S;
demeton; demeton-O; demeton-S; demeton-methyl; demeton-O-methyl; demeton-S-
methyl; demeton-S-methylsulphon; disulfoton; ethion; ethoprophos; IPSP; isothioate;
malathion; methacrifos; oxydemeton-methyl; oxydeprofos; oxydisulfoton; phorate;
sulfotep; terbufos; thiometon; amidithion; cyanthoate; dimethoate; ethoate-methyl;
formothion; mecarbam; omethoate; prothoate; sophamide; vamidothion chlorphoxim;
phoxim; phoxim-methyl; azamethiphos; coumaphos; coumithoate; dioxathion;
endothion; menazon; morphothion; phosalone; pyraclofos; pyridaphenthion;
quinothion; dithicrofos; thicrofos; azinphos-ethyl; azinphos-methyl; dialifos; phosmet;
isoxathion; zolaprofos; chlorprazophos; pyrazophos; chlorpyrifos; chlorpyrifos-
methyl; butathiofos; diazinon; etrimfos; lirimfos; pirimiphos-ethyl; pirimiphos-
methyl; primidophos; pyrimitate; tebupirimfos; quinalphos; quinalphos-methyl;
athidathion; lythidathion; methidathion; prothidathion; isazofos; triazophos;
azothoate; bromophos; bromophos-ethyl; carbophenothion; chlorthiophos; cyanophos;
cythioate; dicapthon; dichlofenthion; etaphos; famphur; fenchlorphos; fenitrothion;
fensulfothion; fenthion; fenthion-ethyl; heterophos; jodfenphos; mesulfenfos;
parathion; parathion-methyl; phenkapton; phosnichlor; profenofos; prothiofos;
sulprofos; temephos; trichlormetaphos-3; trifenofos; butonate; trichlorfon;
mecarphon; fonofos; trichloronat; cyanofenphos; EPN; leptophos; crufomate;
fenamiphos; fosthietan; mephosfolan; phosfolan; pirimetaphos; acephate;
isocarbophos; isofenphos; methamidophos; propetamphos; dimefox; mazidox;
mipafox
Oxadiazine Insecticides
indoxacarb
Phthalimide Insecticides
dialifos; phosmet; tetramethrin
Pyrazole Insecticides
acetoprole; ethiprole; fipronil; tebufenpyrad; tolfenpyrad; vaniliprole
Pyrethroid Insecticides
acrinathrin; allethrin; bioallethrin; barthrin; bifenthrin; bioethanomethrin; cyclethrin;
cycloprothrin; cyfluthrin; beta-cyfluthrin; cyhalothrin; gamma-cyhalothrin; lambda-
cyhalothrin; cypermethrin; alpha-cypermethrin; beta-cypermethrin; theta-
cypermethrin; zeta-cypermethrin; cyphenothrin; deltamethrin; dimefluthrin;
dimethrin; empenthrin; fenfluthrin; fenpirithrin; fenpropathrin;
fenvalerate; esfenvalerate; flucythrinate; fluvalinate; tau-fluvalinate; furethrin;
imiprothrin; metofluthrin; permethrin; biopermethrin; transpermethrin; phenothrin;
prallethrin; profluthrin; pyresmethrin; resmethrin; bioresmethrin; cismethrin;
tefluthrin; terallethrin; tetramethrin; tralomethrin; transfluthrin; etofenprox;
flufenprox; halfenprox; protrifenbute; silafluofen
Pyrimidinamine Insecticides
flufenerim; pyrimidifen
Pyrrole Insecticides
chlorfenapyr
Tetronic Acid Insecticides
spiromesifen
Thiourea Insecticides
diafenthiuron
Urea Insecticides
flucofuron; sulcofuron
Other Insecticides
closantel; clorpyrifos, crotamiton; EXD; fenazaflor; fenoxacrim; hydramethylnon;
isoprothiolane; malonoben; metoxadiazone; niflundide; pyridaben; pyridalyl;
rafoxanide; triarathene; triazamate

Examples of bactericides are shown in Table 3:

TABLE 3
Bactericides
bronopol; 2-(thiocyanatomethylthio) benzothiazole (busan),
cresol; dichlorophen; dipyrithione; dodicin; fenaminosulf;
formaldehyde; hydrargaphen; 8-hydroxyquinoline sulfate;
kasugamycin; nitrapyrin; octhilinone; oxolinic acid; oxytetracycline;
probenazole; streptomycin; tecloftalam thiomersal, Isothiazolone-
type bactericides such as, for example, Kathon 930, Kathon WT,
Methylisothiazolinone, Benzisothiazolin-3-one and 2-octyl-3-
isothiazolone.

Preferred Bactericides Include: bronopol; cresol; dichlorophen; dipyrithione; dodicin; fenaminosulf; formaldehyde; hydrargaphen; 8-hydroxyquinoline sulfate; kasugamycin; nitrapyrin; octhilinone; oxolinic acid; oxytetracycline probenazole; streptomycin; tecloftalam; thiomersal.

The particles are preferably dispersed in a dispersant, such as acrylic copolymers, aqueous solution of copolymers with pigment affinity groups, modified polyacrylate, acrylic polymer emulsions, modified lignin and the like. If desired, a stabilizer as is known in the art can be used.

Inorganic metal compounds having biocidal activity, such as compounds of copper, tin, silver, nickel, etc, can also be used in combination with micronized organic biocide formulations. For example, non-limiting copper based fungicides or insecticides include cuprous oxide, cupric oxide, copper hydroxide, copper carbonate, basic copper carbonate, copper oxychloride, copper 8-hydroxyquinolate, copper dimethyldithiocarbamate, copper omadine, and copper borate.

The micronized organic biocides can be mixed with other water soluble biocides, such as quaternary ammonium compounds. Such compounds have the following structure:

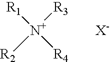

where R1, R2, R3, and R4 are independently selected from alkyl or aryl groups and X⁻ selected from chloride, bromide, iodide, carbonate, bicarbonate, borate, carboxylate, hydroxide, sulfate, acetate, laurate, or any other anionic group. Preferred quaternary ammonium compounds include alkyldimethylbenzylammonium chloride, alkyldimethylbenzylammonium carbonate/bicarbonate, dimethyldidecylammonium chloride and dimethyldidecylammonium carbonate/bicarbonate.

The composition of the present invention may additionally comprise non-biocidal components to further enhance the performance of the micronized organic biocide formulation or the appearance and performance of the resulting treated wood products. Non-limiting examples of such non-biocideal components are water repellants (for example, wax emulsions), colorants, emulsifying agents, dispersants, stabilizers, UV inhibitors, wood dimensional stabilizers, enhancing agents which improve the bio-efficacy of micronized organic biocides (such as trialkylamine oxides and alkoxylated diamines) and the like. Those skilled in the art will recognize that some of these agents, while included in the composition primarily for reasons other than biocidal ability, may also have some biocidal properties.

Enhancing agents such as trialkylamine oxides, can be included in the compositions of the present invention. Preferred trialkylamine oxides have the following structure: 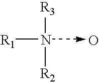
where R₁ is a linear or cyclic C₈ to C₄₀ saturated or unsaturated group and R₂ and R₃ independently are linear C₁ to C₄₀ saturated or unsaturated groups.

Alkoxylated diamines can also be included in the composition of the present invention as enhancing agents. Preferred alkoxylated diamines have the following structure: 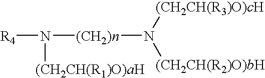
where n is an integer from 1 to 4, R₁, R₂ and R₃ are independently selected from the group consisting of hydrogen, methyl, ethyl and phenyl; and a, b and c are each integers from 1 to 6; and R₄ is fatty alkyl group having in the range of from 8 to 22 carbons.

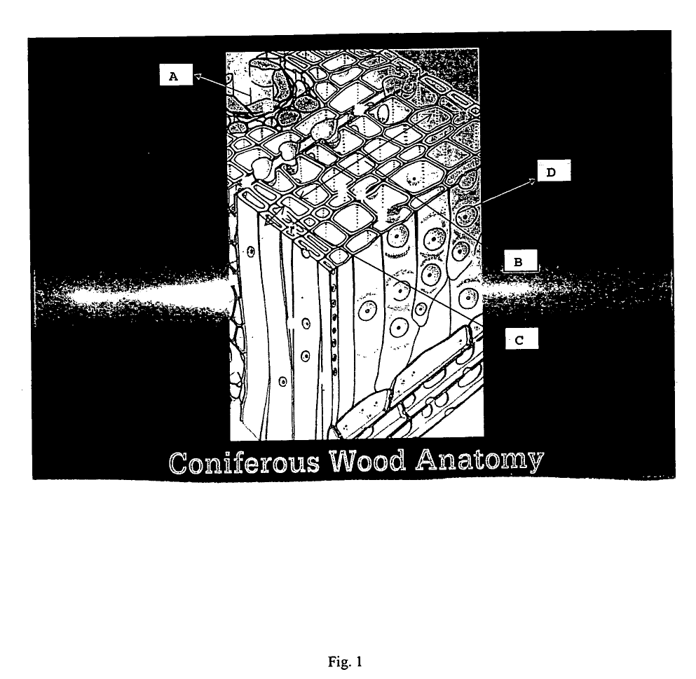

Without desiring to be bound by theory, penetration of the micronized dispersion formulation into wood takes place because particles migrate into or are taken up by tracheids in the wood. FIG. 1 shows the physiological structure of wood. As shown in FIG. 1, the primary entry and movement of fluids through wood tissue occurs primarily through the tracheids and border pits. Fluids are transferred between wood cells by means of border pits. Wood tracheids generally have diameters of around 30 microns, and thus good penetration can be achieved by the use of particles having long axis dimensions (“particle size” which are less than the tracheid diameters of the wood or wood product to be treated. Particles having diameters which are larger than the average diameter of the tracheids will generally not penetrate the wood (i.e., they will be “filtered” by the wood) and may block, or “clog” tracheids from taking in additional particles.

The diameter of the tracheids depends upon many factors, including the identity of the wood. As a general rule, if the organic biocides disclosed herein have a particle size in excess of 25 microns, the particles may be filtered by the surface of the wood and thus may not be uniformly distributed within the cell and cell wall.

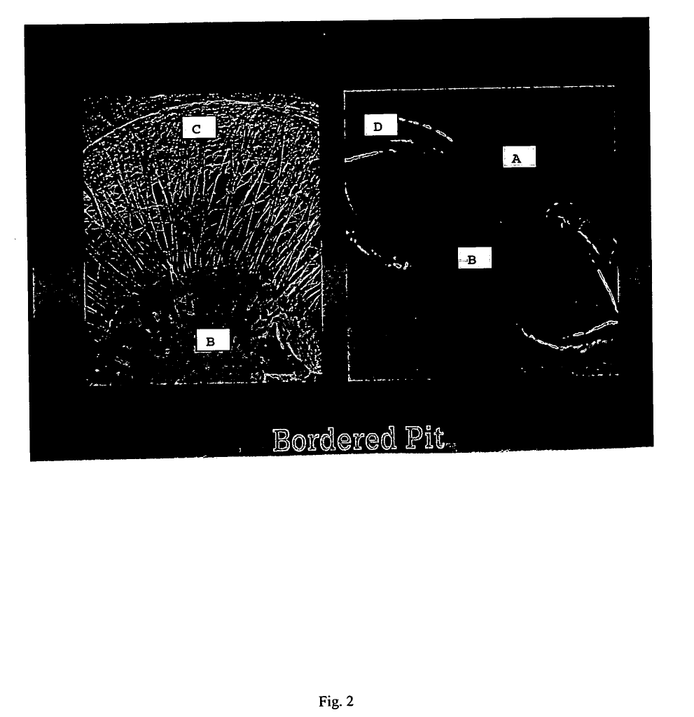

Studies by Mercury-Porosimetry technique indicated that the overall diameter of the border pit chambers typically varies from a several microns up to thirty microns while, the diameter of the pit openings (via the microfibrils) typically varies from several hundredths of a micron to several microns. FIG. 2 depicts the border pit structure for coniferous woods. Thus, the use organic biocide particles with sizes such that the particles can travel through the pit openings will increase penetration and improve the uniformity of distribution of the particulate organic biocide.

In one embodiment particle size of the micronized particles used in the dispersion formulation disclosed herein can be micronized, i.e., with a long axis dimension between 0.001-25 microns. In another embodiment, the particle size is between 0.001-10.0 microns. In another embodiment, the particle size is between 0.01 to 10.0 microns. If superior uniformity of penetration is desired, particle size of the organic biocide used in the dispersion formulation disclosed herein can be between 0.01-1.0 microns.

In addition to a recommended upper limit of 25 microns, Particles which are too small can leach out of the wood over time. It is thus generally recommended that the particulate organic biocide comprise a majority weight percent of particles which have diameters which are not less than 0.001 microns.

Because particles which are too large can clog the wood and particles which are too small can leach from the wood, it is advisable to use particle size distributions which contain relatively few particle sizes outside the range of 0.001 to 25 microns. It is preferred that no more than 20 weight percent of the particles have diameters which are greater than 25 microns. Because smaller particles have an increased chance of leaching from the wood, it is also preferred that no more than 20 wt % of the particles have diameters under 0.001 microns. Regardless of the foregoing recommendations, it is generally preferred that greater than 80 wt % of the particles have a diameter in the range of 0.001 to 25 microns. In more preferred embodiments, greater than 85, 90, 95 or 99 wt percent particles are in the range of 0.001 to 25 microns.

For increased degree of penetration and uniformity of distribution, at least 50 wt % of the particles should have diameters which are less than 10 microns. More preferred are particle distributions which have at least 65 wt % of the particles with sizes of less than 10 microns. In an additional embodiment, less than 20 wt % of the particles have diameters of less than 1 micron.

The present invention also provides a method for preservation of wood. In one embodiment, the method comprises the steps of treating wood with a composition (treating fluid) comprising a dispersion of micronized organic biocides. In another embodiment, wood is treated with a composition comprising a dispersion of micronized organic biocides and a water soluble biocides. The size of the micronized particles of organic biocide is between 0.001 to 25 microns, preferably between 0.001 to 10 microns, more preferably between 0.01 to 10 microns and most preferably between 0.01 to 1.0 microns.

In another embodiment, the wood is treated with a composition comprising soluble metal biocidal compounds and micronized organic biocides.

The treating fluid may be applied to wood by dipping, soaking, spraying, brushing, or any other means well known in the art. In a preferred embodiment, vacuum and/or pressure techniques are used to impregnate the wood in accord with this invention including the standard processes, such as the “Empty Cell” process, the “Modified Full Cell” process and the “Full Cell” process, and any other vacuum and/or pressure processes which are well known to those skilled in the art.

The standard processes are defined as described in AWPA Standard C1-03 “All Timber Products—Preservative Treatment by Pressure Processes”. In the “Empty Cell” process, prior to the introduction of preservative, materials are subjected to atmospheric air pressure (Lowry) or to higher air pressures (Rueping) of the necessary intensity and duration. In the “Modified Full Cell”, prior to introduction of preservative, materials are subjected to a vacuum of less than 77 kPa (22 inch Hg) (sea level equivalent). A final vacuum of not less than 77 kPa (22 inch Hg) (sea level equivalent) should be used. In the “Full Cell Process”, prior to introduction of preservative or during any period of condition prior to treatment, materials are subjected to a vacuum of not less than 77 kPa (22 inch Hg). A final vacuum of not less than 77 kPa (22 inch Hg) is used.

The following examples are provided to further describe embodiments of the disclosure but are in no way limiting to the scope of disclosure. Examples 1 through 6 demonstrate the formulation of the concentrated dispersions of organic biocides. Examples 7 through 15 demonstrate the preparation of treating fluids using concentrated dispersions for the treatment of wood.

The invention is further described through the following examples which are intended to be illustrative and not restrictive in any way.

### EXAMPLE 1

500 grams of cyproconazole powder is added to a container containing 825 grams of water and 175.0 grams of a commercially available dispersant. The mixture is mechanically stirred for 5 minutes and then placed in a grinding mill. The sample is ground for about 90 minutes, and a stable dispersion containing about 33.3% wt % cyproconazole is obtained with an average particle size of 0.20 micrometers.

### EXAMPLE 2

1000 grams of C powder is mixed with 2600.0 grams of water and 400.0 grams of dispersants. The mixture was mechanically stirred for 10 minutes. The mixture was then placed in a grinding mill and ground for about 140 minutes. A stable dispersion is obtained with roughly 100% particles less than one micrometer.

### EXAMPLE 3

500.0 grams of imidachloprid powder is mixed with 966.7 grams of water and 200.0 grams of wetting agents/dispersants. The mixture was mechanically stirred for about 10 minutes. The mixture is then placed in a grinding mill and ground for about 180 minutes. A stable dispersion containing approximately 30.0% wt % imidachloprid is obtained with an average particle size of 0.30 micrometers.

### EXAMPLE 4

500 grams of cyproconazole powder and 500 grams of imidachloprid are mixed with 1550 grams of water and 450 grams of dispersants. The mixture is mechanically mixed for about 15 minutes and placed in a grinding mill. The mixture is ground for about 260 minutes and a stable dispersion containing about 16.7% cyproconazole and 16.7% wt % imidachloprid is obtained with an average particle size of 0.35 micrometers.

### EXAMPLE 5

1000 grams of propiconazole powder and 200 grams of bifenthrin are mixed with a mixture of 2500 grams water and 300 grams dispersant. The mixture is mechanically mixed for about 20 minutes and then added to a grinding mill. The mixture is ground for about 160 minutes and a stable dispersion is obtained with 100% particles less than one micrometers.

### EXAMPLE 6

500.0 grams of cyproconazole powder and 250.0 grams of fipronil powder are added to a 4000 ml beaker which contains about 1350.0 grams of water and 400.0 grams of dispersant. The mixture is allowed to mix for 30 minutes prior to adding to a grinding media mill. The mixture is ground for 290 minutes and a stable dispersion with 30.0% wt % solid is obtained with an average particle size of 0.35 micrometers.

### EXAMPLE 7

One gram of cyproconazole dispersion from Example 1 is with 3000 grams of water to produce a preservative treating fluid containing 0.0 1% wt % cyproconazole. The fluid is then used to treat 2″×4″×10″ samples of southern pine sapwood, end sealed with epoxy resin, using an initial vacuum of 28″ Hg for 15 minutes, followed by a pressure cycle of 115 psi for 25 minutes and a final vacuum of 27″ Hg for 10 minutes. The resulting treated wood is weighed and found to have doubled its weight. The treated sample is cut and the cross section is taken and submitted scanning electron microscopic analysis. The sample is found to a complete particle penetration through the whole cross section and a uniform distribution of particle.

### EXAMPLE 8

One gram dispersion from Example 1 and one gram dispersion from Example 3 are added to 3000 grams of water. The mixture is allowed to mix for 10 minutes. The resulting fluid is used to 2″×4″×10″ samples of southern pine sapwood, end sealed with epoxy resin, using an initial vacuum of 28″ Hg for 15 minutes, followed by a pressure cycle of 120 psi for 30 minutes and a final vacuum of 27″ Hg for 10 minutes. The resulting treated wood is weighed and found to have doubled its weight.

### EXAMPLE 9

4000 grams of treating fluid containing 0.05% wt % of tebuconazole and 0.0075% wt % imidachloprid is prepared by mixing tebuconazole dispersion from Example 2 and imidachloprid from Example 3 with water.

A southern pine stake measuring 1.5″×3.5″×10″ is placed in a laboratory retort with a vacuum of 27″ Hg for 15 minutes. The above treating fluid is then pumped into the retort and the retort pressurized to 130 psi for 30 minutes. The solution is drained from the retort and the test stake weighed. Based on the weight pickup, the test stake doubles its weight and SEM indicates the uniform particle penetration and distribution.

### EXAMPLE 10

4000 grams of treating fluid containing 0.05% wt % propiconazole and 0.010% wt % bifenthrin is prepared by adding the dispersion from Example 5 to water. The mixture is mechanically mixed for about 10 minutes and then pumped to a treating retort where a southern pine stake measuring 1.5″×3.5″×10″ is pre-vacuumed under 27″ Hg for 10 minutes. The retort is then pressurized to 100-120 psi for about 20 minutes. The solution is drained from the retort and the test stake weighed.

### EXAMPLE 11

A preservative treating formulation is prepared by adding 0.15 kg of dispersion from Example 4 and 0.10 kg dispersion from Example 2 to 25.0 kg of water. This fluid is allowed to mix until a homogenous fluid is prepared. This fluid was used to treat southern pine samples measuring at 1.5″×5.5″×48″ by the full-cell process. The weight of the treated samples double and demonstrate a uniform distribution of particles throughout the wood cells and is found to be resistant to decay and insect attack.

### EXAMPLE 12

A preservative treating composition is prepared by adding 2.0 grams of dispersion from Example 6 to 5.0 kg of water. The resulting fluid contains about 0.08% wt % cyproconazole and 0.04% wt % fipronil. This fluid is then used to treat southern pine measuring 1.5″×3.5″×10″ using the full-cell process wherein the wood is initially placed under a vacuum of 30″ Hg for 30 minutes, followed by the addition of the treating solution. The system is then pressurized for 30 minutes at 100 psi. A final vacuum of 28″ Hg for 30 minutes is applied to the wood to remove residual liquid. The wood is found to contain a uniform distribution of preservative particle throughout the cross sections and is resistant to fungal and insect attack.

### EXAMPLE 13

5 kg preservative treating composition is prepared by mixing dispersion concentrate from Example 5 and dimethyldidecylammonium bicarbonate/carbonate (DDAC). The concentration of propiconazole, bifenthrin and DDAC in the final fluid is 0.05% wt %, 0.01% wt % and 0.50% wt %, respectively. This fluid is then used to treat southern pine measuring 1.5″×3.5″×10″ using the full-cell process wherein the wood is initially placed under a vacuum of 30″ Hg for 30 minutes, followed by the addition of the treating solution. The system is then pressurized for 30 minutes at 100 psi. A final vacuum of 28″ Hg for 30 minutes is applied to the wood to remove residual liquid. The wood is found to contain a uniform distribution of preservative particle throughout the cross sections and is resistant to fungal and insect attack.

### EXAMPLE 14

A preservative treating composition containing 0.01% wt % cyproconazole, 0.01% wt % imidachloprid and 0.25% wt % Cu is prepared by mixing dispersion concentrate from Example 4 and copper monoethanolamine solution (Cu-MEA); This fluid is then used to treat southern pine measuring 1.5″×3.5″×10″ using the full-cell process wherein the wood is initially placed under a vacuum of 30″ Hg for 30 minutes, followed by the addition of the treating solution. The system is then pressurized for 30 minutes at 100 psi. A final vacuum of 28″ Hg for 30 minutes is applied to the wood to remove residual liquid. The wood is found to contain a uniform distribution of preservative particle throughout the cross sections and is resistant to fungal and insect attack.

### EXAMPLE 15

A preservative composition containing 0.02% wt % tebuconazole and 0.50% wt % N,N-dimethyl-1-hexadecylamine-N-oxide was prepared by mixing dispersion concentrate from Example 2 and 30% N,N-dimethyl-1-hexadecylamine-N-oxide solution. This fluid is then used to treat southern pine measuring 1.5″×3.5″×10″ using the full-cell process wherein the wood is initially placed under a vacuum of 30″ Hg for 30 minutes, followed by the addition of the treating solution. The system is then pressurized for 30 minutes at 100 psi. A final vacuum of 28″ Hg for 30 minutes is applied to the wood to remove residual liquid. The wood is found to contain a uniform distribution of preservative particle throughout the cross sections and is resistant to fungal and insect attack.

1. A wood preservative composition comprising a particulate organic biocide, wherein greater than 80 weight percent of the biocide particles have diameters in the range of 0.001 microns to 25 microns.

2. A wood preservative composition as in claim 1, wherein the organic biocide is selected from the group consisting of the list in Tables 1, 2 and 3.

3. A wood preservative composition as in claim 1, wherein fewer than 20 weight percent of the particles have a diameter of greater than 25 microns.

4. A wood preservative composition as in claim 1 wherein fewer than 20 weight percent of the particles have a diameter of less than 0.001 microns.

5. A wood preservative composition as in claim 1, wherein greater than 50 weight percent of the biocide particles have diameters in the range of 0.01 microns to 10 microns.

6. A wood preservative composition as in claim 3, wherein greater than 80 weight percent of the particles have diameters which are less than 1 micron.

7. A wood preservative composition as in claim 1, wherein the composition comprises an enhancing agent.

8. A wood preservative composition as in claim 7, wherein the enhancing agent is selected from the group consisting of a trialkylamine oxide or an alkoxylated diamine.

9. A wood preservative composition as in claim 1, wherein greater than 85, 90, 95 or 99 weight percent of the biocide particles have diameters in the range of 0.001 microns to 25 microns.

10. A wood preservative composition of claim 1, wherein the organic biocide is selected from the group consisting of cyproconazole, tebuconazole, imidachloprid, propiconazole, bifenthrin, fipronil, dimethyl didecyl ammoniam bicrobonate/carbonate and N,N-dimethyl-1-hexadecylamine-N-oxide.

11. A process for preserving wood, said process comprising:
a) providing a composition comprised of particulate organic biocide comprised of particles having diameters in the range of 0.001 to 25 microns;
b) applying said composition to wood such that at least some of said particles penetrate the surface of the wood.

12. A process as in claim 11, wherein the organic biocide is selected from the group consisting of the list in Tables 1, 2 and 3.

13. A process as in claim 11 wherein fewer than 20 weight percent of the particles have a diameter of greater than 25 microns.

14. A process as in claim 11 wherein fewer than 20 weight percent of the particles have a diameter of less than 0.005 microns.

15. A process as in claim 11 wherein at least 50 weight percent of the biocide particles have diameters in the range of 0.01 microns to 1 micron.

16. A process as in claim 11 wherein greater than 80 weight percent of the particles have diameters which are less than 1 micron.

17. A process as in claim 11, wherein greater than 85, 90, 95 or 99 weight percent of the biocide particles have diameters in the range of 0.001 microns to 25 microns.

18. A process as in claim 11, wherein the organic biocide is selected from the group consisting of cyproconazole, tebuconazole, imidachloprid, propiconazole, bifenthrin, fipronil, dimethyl didecyl ammoniam bicrobonate/carbonate and N,N-dimethyl-1-hexadecylamine-N-oxide.

19. Wood comprising particulate organic biocide which is inside the wood, wherein at least some of the particles of said organic biocide have diameters in the range of from 0.001 to 25 microns.

20. Wood as in claim 19, wherein the organic biocide is selected from the group consisting of the list in Tables 1, 2 and 3.

21. Wood as in claim 19 which additionally comprises an enhancing agent.

22. Wood as in claim 21, wherein the enhancing agent is selected from the group consisting of an alkoxylated diamine and a trialkylamine oxide.

23. Wood as in claim 19 wherein the organic biocide is selected from the group consisting of cyproconazole, tebuconazole, imidachloprid, propiconazole, bifenthrin, fipronil, dimethyl didecyl ammoniam bicrobonate/carbonate and N,N-dimethyl-1-hexadecylamine-N-oxide.
